(19) United States
(12) Patent Application Publication
(10) Pub. No.: US 2026/0294822 A1
(43) Pub. Date: Oct. 1, 2026

(54) COMPOSITIONS AND METHODOLOGY FOR DELIVERY OF ALPHA-GALACTOSYLCERAMIDE COMPOUNDS

(71) Applicant: EnGeneIC Molecular Delivery Pty Ltd, Sydney (AU)

(72) Inventors: Himanshu Brahmbhatt, Killara, Sydney (AU); Jennifer MacDiarmid, Glebe, Sydney (AU)

(73) Assignee: EnGeneIC Molecular Delivery Pty Ltd, Sydney (AU)

(21) Appl. No.: 19/474,956

(22) PCT Filed: Apr. 16, 2024
(86) PCT No.: PCT/IB2024/053714

§ 371 Date: Oct. 13, 2025

Related U.S. Application Data
(60) Provisional application No. 63/459,940, filed on Apr. 17, 2023.

(51) Int. Cl. A61K 9/50; A61K 31/7032; A61K 39/00; A61K 39/39

(52) U.S. Cl. CPC A61K 9/5068; A61K 31/7032; A61K 39/39; A61K 2039/55511

(57) ABSTRACT

Compositions and methods are described for treating a disease, such as cancer or a viral or bacterial infection. More specifically, the provided compositions comprise encapsulated CD Id-restricted invariant Natural Killer T cell antigen, a bacterial α-galactosylceramide.

WO

### CROSS-REFERENCE TO RELATED APPLICATIONS

This application claims the benefit under 35 U.S.C. § 119(e) of U.S. Provisional Patent Application No. 63/459,940, filed Apr. 17, 2023, the entire contents of which is incorporated herein by reference in its entirety.

### BACKGROUND

The present technology relates generally to the field of preparing and delivering a type of α-galactosylceramide molecule for therapeutic purposes.

The cerebrosides belong to the family of glycosphingolipids (GSLs) and are important components of a wide variety of tissues, organs, and nerve cell membranes in biological systems. Cerebrosides consist of a ceramide part, with two long fatty chains, linked to a polar residue (single sugar or polysaccharide).

Among the best known members of the GSL family in higher organisms, including humans, are the galactoceramides (GalCers), comprised of a D-galactose (Gal) residue linked by a β1-1′-glycosidic bond to a ceramide (Cer) composed of D-erythro-sphingosine and long-chain fatty acid. Fatty acids attached to the sphingosine vary in length (C₁₄-C₂₆), with stearic acid (C₁₈) being the most abundant. The galactosylceramides are enriched in long chain α-hydroxy fatty acids (C₁₈-C₂₆).

Among other lipids, GSLs are able to induce cell-mediated immunity when they are presented to the subset of T lymphocytes called Natural Killer T (NKT) cells, characterized by the expression of markers typical for T lymphocytes as well as NK cells. NKT cells are further divided into type I or invariant natural killer (INKT) cells and type II or non-iNKT cells.

iNKT cells recognize and bind lipid antigens and are characterized by a restricted repertoire of T-cell receptors (TCRs). A lipid antigen is recognized and bound by the INKT TCR only when the antigen is presented by the monomorphic HLA class I-related molecule CD1 protein molecule, expressed by antigen-presenting cells (APCs). In humans, CDI isoforms are divided into group I, represented by CD1a, CD1b, CD1e and CD1e, and group II, represented by CD1d. Each CD1 binds a defined set of GSLs, has tissue-specific expression, and presents antigens to T cells with a specific repertoire of TCRs.

At Kirin Brewery, screening of marine natural products for anti-tumor activities led Koezuka and coworkers to the discovery of “agelasphins,” a class of galactosylceramide glycolipids, which they isolated from Agelas mauritianus, the Okinawan marine sponge. Found to cause lymphocyte proliferation, the agelasphins contained α-glycosidic bonds between the sugar and ceramide. The latter feature contrasted with the β-glycosidic linkages that typify glycosylceramides found in higher organisms.

FIG. 1 depicts a member of the class, agelasphin 9b, with ceramide numbering shown. An extension of the acyl chain length in agelasphin 9b by two carbons led to KRN7000 (Wieland Brown, 2013), a synthetic α-galactosylceramide (α-GalCer) also shown in FIG. 1. Relative to the original agelasphins, KRN7000 lacks a hydroxyl group at the 2 position on the fatty acyl chain. As FIG. 1 shows, KRN7000 also possesses a 4-hydroxyl group on the sphingosine chain, which is absent in some of the agelasphins.

Presented by CD1d of APCs, KRN7000 has proven a notable stimulator of iNKT cells. The interaction of a presented lipid-CD1d complex with the T cell receptor (TCR) of iNKT cells can form a stable trimeric complex (CD1d::lipid-TCR), which is a prerequisite to activating those cells and triggering the release of a variety of signaling molecules, cytokines and chemokines, involved in cellular communication (Kronenberg, 2005; Vartabedian et al., 2016). Thus, upon presentation of KRN7000 by CD1d-expressing APCs, INKT are activated and rapidly produce large amounts of predominantly IFN-γ but also IL-4, cumulatively resulting in the activation of NK cells, CD4+ and CD8+ T cells, B cells, neutrophils, macrophages and the dendritic cells with which they interact to fight against tumors and to effect antimicrobial functions (Th1) or to protect against autoimmune diseases (Th2).

The therapeutic potential suggested by preclinical studies of KNR7000 (Nakagawa et al., 1998; Hayakawa et al., 2001; Schneiders et al. 2011a) has not been born out in clinical experience with this glycolipid. During an initial phase 1 study in patients with refractory solid tumors (Giaccone et al., 2002), intravenous (i.v.) administration of KRN7000 temporarily reduced the number of circulating iNKT cells for a least one week and increased GM-CSF and TNF-α levels, but only in patients with a relatively high pre-treatment iNKT level, and there was no significant clinical effect overall (Waldowska et al., 2017). Similar results pertained in other clinical trials, where a lack of immunoreactivity was observed after i.v. administrations of KRN 7000 (Schneiders et al., 2011a, b). Although immunological, biochemical, and even some clinical responses were observed in patients treated in KRN7000, the results generally lacked consistency (Schneiders et al., 2011b).

The effectiveness of KNR7000 has been thought to be constrained by the opposing effects of the Th1 cytokines (IFN-γ) and Th2 cytokines (IL-4) induced by this glycolipid. In order to develop selective Th1 or Th2 activators, therefore, many α-GalCer analogs have been synthesized (Blauvelt et al., 2008; Chang et al., 2007; Chiba et al., 2004; Fujio et al., 2006; Hung et al., 2007; Kaieda et al., 2007; Liang et al., 2008; Velmourougane et al., 2009; Wu et al., 2006), and their immune-modulating activities were shown to be related to the affinity of binding to CD1d (Fujio et al., 2006).

For example, studies using a CD1d array have established that the secretion of IFN-γ and IL-4 by iNKT cells is determined by the binding of α-GalCer analogs to CD1d (Liang et al., 2008), and a higher affinity for CD1d would shift the cytokine release profile toward a stronger Th1 response (Chang et al., 2007; Liang et al., 2008). Furthermore, α-GalCer analogs with various aryl moieties at the acyl chain terminus have shown strong iNKT activation with a strong Th1 bias. One such derivative, 7DW8-5, showed a superior adjuvant activity relative to that of other α-GalCer analogs when tested with malaria and HIV antigens in mice (Padte et al., 2013). In another α-GalCer derivative, a 1,2,3-triazole moiety replaces an amide linkage in the α-GalCer scaffold, resulting in an increase in the IL-4 versus IFN-γ bias of released cytokines (Lee et al., 2007).

As noted, KRN7000 is a synthetic derivative of agelasphin 9b, a glycolipid isolated from Agelas mauritianus, a marine sponge, in the 1990s (Banchet-Cadeddu et al., 2011). Now as then, it is unclear why a sponge glycolipid could be basis for so strong an activator of mammalian iNKT cells as KRN7000.

One possibility is suggested by the discovery that two glycosphingolipids, resembling the chemical structure of KRN 7000, are produced by Sphingomonas species, Gram-negative bacteria that lack lipopolysaccharide (LPS). More specifically, α-glucuronosylceramide (GSL-1) was isolated from the cell wall of Sphingomonas capsulate, whereas α-galacturonosylceramide (GSL-1′) was isolated from S. yanoikuyae and S. wittichii (Tsuji, 2006).

The abundant presence of a family of monoglycosylceramides in the cell wall of Sphingomonas species may substitute for LPS. In any event, both GSL-1 and GSL-1′ have been shown to stimulate murine and human NKT cells in vitro, and flow-cytometric data indicate that both glycolipids actually bind to CD1d molecules and are recognized by NKT cells (id).

Relative to KRN7000, the glucuronic acid and the galacturonic acid of GSL-1 and GSL-1′, respectively, have the carboxyl group at the 6-carbon position. Moreover, the Sphingomonas-derived glycosphingolipids possess a hydroxyl group at the 2-carbon position in the fatty-acyl chain, thus more closely resembling the original agelasphins of the marine sponge. Additionally, the absence of a 4-hydroxyl group at the sphingosine chain of the Sphingomonas-derived glycosphingolipids is mirrored in the structure of some of the agelasphins. Generally, see Tsuji, 2006.

These observations take on added significance in light of the discovery of Sphingomonas alaskensis (a/k/a Sphingopyxis alaskensis) in high abundance in the oligotrophic Pacific waters near the southern part of Japan. Accordingly, it may well be that the agelasphins are produced not by A. mauritianus sponges themselves but rather by Sphingomonas-like bacteria that are either bound to or ingested by and contained within the sponges.

In a similar vein, uncertainty persists over whether natural contact of mammalian organisms with KRN7000 occurs by virtue of endogenous production or microbial sources. Although KRN7000 has been shown to be an activator of NKT cells and a mediator of immune response in some cancers or virus infections, as noted above (Ko et al., 2005), KRN7000 is not produced by mammals. In addition, evidence is lacking that β-GalCer is able to provoke an immune reaction.

On the other hand, the bacterium Bacteroides fragilis, a commensal of the human gut, has been demonstrated to produce an α-GalCer molecule, α-GalCer_{Bf}, that is a natural ligand for CD1d-mediated iNKT cell activation, with immunological properties similar to those of KRN7000 (Wieland Brown et al., 2013). Based on this work, two more strains of the human gut microbiome, Bacteroides vulgatus and Prevotella copri, have been shown to produce an α-GalCer molecule (von Gerichten et al., 2017). Furthermore, an α-GalCer molecule isolated from murine large intestine, hence called α-GalCer_{MLI}, has been demonstrated to be of bacterial origin, likely from a Bacteroides source (von Gerichten et al., 2019).

Accordingly, there is an understanding that gut bacteria-derived sphingolipids, including a variety of α-GalCer molecules produced by Bacteroides and other commensal species, play important roles in mammalian physiology, such as maintaining intestinal homeostasis. See Brown et al., 2019, for instance. Yet there also are data indicating that the bacterial α-GalCer molecules do not activate iNKT cells as strongly as KRN7000, the synthetic α-GalCer agonist that has represented a standard in glycolipid research.

Thus, Weiland-Brown et al. (2013), supra, have noted subtle but important structural differences between α-GalCer_{Bf} and KRN7000 indicating that the former may be a less potent immune activator. Moreover, the IL-2 production induced by α-GalCer (d18:0/βh16:0), a synthetic analog of α-GalCer_{MLI}, was comparable to that of α-GalCer_{Bf} but lower than the IL-2 production induced by KRN 7000, which contains the very long chain cerotic acid (26:0) (von Gerichten et al., 2017). Similar to KRN7000, α-GalCer (d18:1/24:1) with the very long chain nervonic acid (24:1) activated iNKT cells at lower concentrations than the other tested α-GalCers, including α-GalCersBF, which generally comprised shorter C16- and C17-fatty acids. See FIG. 5 of von Gerichten et al., 2019.

### SUMMARY

As described above, the use of KRN7000 in clinical therapy has been limited, in part because the synthetic α-GalCer agonist induces NKT cells to produce both Th1 and Th2 cytokines at the same time in large quantities. Additionally, bacterial α-GalCer molecules have tested no better than or inferior to KRN7000 in relation to activating iNKT cells. It is not surprising, therefore, that the scientific literature does not place any bacterial α-GalCer molecule in a therapeutic context; nor does it suggest, for purposes of a medicament, the production in bulk quantities of pharmaceutical-grade bacterial α-GalCer compound.

By contrast, the present disclosure provides a composition comprising (a) a pharmaceutically acceptable carrier and (b) a plurality of delivery elements selected from intact bacterially derived minicells, intact killed bacterial cells, intact mammalian cells, and extracellular vesicles, which delivery elements comprise an α-galactosylceramide compound that (i) comprises (A) a N-acyl chain of 16 to 17 carbons in length and (B) a hydroxyl group on the β-rather than the α-carbon and (ii) does not present a 4-hydroxy group on its sphingoid base, wherein the compound can form a CD1d-lipid-TCR trimeric complex in vivo such that iNKT cells are activated. In some embodiments, the compound further comprises (iii) an iso-branched lipid terminus.

In some embodiments, at least some of the delivery elements are mammalian cells selected from the group consisting of natural killer cells, dendritic cells, and macrophages. In some embodiments, at least some of the delivery elements are extracellular vesicles. In some embodiments, at least some of the delivery elements are intact bacterially derived minicells.

In some embodiments, the compound was purified to pharmaceutical-grade after in vitro expression thereof by commensal bacteria. In some embodiments, the compound was purified to a level in a range from about 90% to about 99%, or greater than about 99%.

In some embodiments, the bacterium is selected from the group consisting of Bacteroides fragilis, Bacteroides vulgatus, and Prevotella copri. In some embodiments, the bacterium is Bacteroides fragilis.In some embodiments, the compound is α-GalCer_{Bf}.

In another aspect, the present disclosure provides a dosage form comprising a single-use vial that comprises about 1×10¹⁰ to about 1×10¹¹ minicells packaged with an α-galactosylceramide compound as described above, such that the vial comprises a therapeutically effective dose of the compound.

In yet another aspect, the present disclosure provides a method of treating or vaccinating against a viral or bacterial infection, comprising administering to a subject in need of such treating or vaccinating a therapeutically effective amount of a composition according to any of the above-enumerated embodiments.

In a further aspect, the present disclosure provides of treating cancer, comprising administering to a subject who suffers from a cancer pathology a therapeutically effective amount of a composition according to any of the above-enumerated embodiments.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows the chemical structures of various ceramide molecules, with carbon numbering shown.

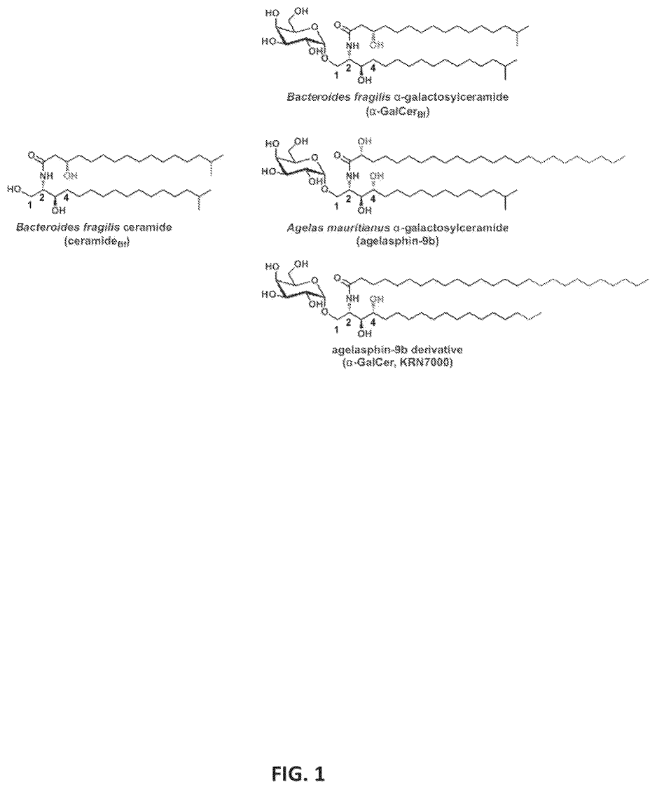

FIG. 2 is a flowchart describing exemplary methodology of glycolipid extraction and purification.

### DETAILED DESCRIPTION

### I. Overview

The foregoing background narrative sets out the considerations that informed an understanding in the field that analogs of KNR7000 were needed, rather than KNR7000 itself (and, by extension, any α-GalCer molecule of like or inferior iNKT-activation potency), in order to realize the therapeutic potential implicated by early research with the synthetic α-GalCer agonist. Discoveries and related insights on the part of the present inventors, however, belie the conventional wisdom in this regard, opening the way to the medicaments and associated production methodology of the present invention.

The inventors determined, for example, that when synthetically purified KRN7000 is mixed exogenously with APC/INKT cell co-culture, it fails to be displayed optimally to the TCR of iNKT cells. This is believed to explain at least in part why previous human trials using purified α-GalCer may have failed in terms of tumor regression. That is, in those trials most of the injected α-GalCer would have been degraded in the serum or lymphatic fluids, and little would have been picked up by circulating APCs and displayed via CD1d on the APC cell surface. The result would have been a suboptimal activation of iNKT cells, which in turn would result in a poor anti-tumor response; hence, a misapprehension emerged in the field about α-GalCer immune-potency per se (see more below).

The present inventors also observed that the α-GalCer::CD1d-activated iNKT cells release IFNγ, which triggers increased expression of CD1d molecules in professional phagocytic cells, e.g., macrophages and dendritic cells. The professional phagocytes thus receive a “preparedness” signal to the effect that an invading pathogen or aberrant tumor cell is carrying glycolipid molecules.

The inventors further discovered that other signaling is required for α-GalCer::CD1d-activated iNKT cells to home into the tumor microenvironment. This insight arose when the inventors made an initial intravenous administration of bacterially derived minicells packaged with a cytotoxic drug, PNU-159682 (PNU), and targeted to the tumor cell surface receptor, Epidermal Growth Factor Receptor (EGFR), via attachment of single-chain, bispecific antibodies to the drug-packaged minicells' cell surface O-polysaccharide component of LPS. These PNU-packaged, EGFR-targeted minicells extravasated from the tumor-associated leaky vasculature and entered into the tumor microenvironment. The anti-EGFR targeting antibody on the minicell surface bound to tumor cell surface EGFRs, resulting in micropinocytosis of the minicells into tumor cells. See Brahmbhatt & MacDiarmid, 2022, which is incorporated herein by reference in its entirety.

Within the tumor cells, the minicells were taken into lysosomes and degraded, whereupon release of the cytotoxic drug triggered apoptosis in the tumor cells. This resulted in release from the apoptotic tumor cells of Damage-Associated Molecular Pattern molecules (DAMPs) (Garg et al., 2010; Krysko et al., 2011) such as ATP (Rock and Kono, 2008). The DAMPs constitute a “find me” signal, which attracts the professional phagocytic cells (macrophages and dendritic cells) and the activated iNKT cells to the tumor microenvironment, where the phagocytic cells recognize and engulf the apoptotic tumor cells via of efferocytosis (Henson 2017).

After antigen processing, the phagocytic cells display the tumor cell-associated polypeptide antigens, via the MHC Class I and II molecules, and glycolipid antigens via the MHC Class I-like molecule CD1d. By way of the draining lymph nodes, the tumor antigen-displaying phagocytic cells go into circulation and, primarily in the spleen and liver, display the polypeptide antigens to CD8+ cytotoxic T cells and the glycolipid antigens to iNKT cells.

Accordingly, the present inventors determined that this signaling series is how iNKT cells recognize tumor-associated glycolipid antigens, migrate into the tumor, recognize the glycolipid antigens on tumor cells, and mediate their cytotoxic effector functions. So informed, the inventors realized (A) that the α-GalCer molecule acted as an adjuvant that triggers the above-described pathway whereby fresh iNKT cells recognize tumor-associated glycolipid antigens and (B) that it is these iNKT cells that eventually kill tumor cells, not the ones that were activated via the α-GalCer molecule in the first instance.

For the inventors, these discoveries and insights belied the conventional wisdom that a heightened potency, relative to KNR7000, of one or another α-GalCer analog was required to achieve anti-tumor efficacy.

To validate this new understanding, the inventors carried out experiments using a combination of (i) antibody-targeted, PNU682-packaged minicells with (ii) KRN7000-packaged minicells. In mouse xenograft studies the inventors demonstrated that this combination was sufficient to achieve highly significant anti-tumor efficacy, while administration of either (i) or (ii) alone failed to achieve such efficacy.

Accordingly, the inventors conceived of medicaments and related production methodology focused on a class of α-GalCer compounds that, even if no better than or inferior to KNR7000 in iNKT-activating potency, nevertheless could serve as an adjuvant to achieve significant anti-tumor efficacy, as described above.

Pursuant to the present invention, the class in question encompasses bacterial α-GalCer molecules that can form a CD1d::lipid-TCR trimeric complex in vivo such that iNKT cells are activated. There are conventional assays available to screen candidate bacterial α-GalCer molecules for trimeric complex-forming activity (von Gerichten et al., 2019). One such assay, along lines detailed by Zhu et al., 2015, entails conducting an enzyme-linked immunosorbent assay (ELISA) for IFNγ in supernatant obtained when cells displaying CD1d::glycolipid are co-incubated with iNKT cells. If the TCR from iNKT cells locks onto the CD1d::glycolipid, forming the trimeric complex, then the ELISA yields a positive signal by virtue of the IFNγ secreted by the iNKT cells.

Unlike the α-GalCer analogs discussed in the background narrative, the bacterial α-GalCer molecules of the present invention do not require complicated and costly synthesis.

Instead they can be produced in bulk by fermentation and then purified to pharmaceutical grade for use according to the present invention (see more below).

In this description, the phrases “bacterial α-GalCer molecule,” “bacterial α-GalCer compound,” and “α-GalCer_{B}” are used interchangeably to denote a chemical entity that possesses the typifying structural characteristics of an α-GalCer, as described above in relation to KRN 7000, but that is further characterized by (1) comprising a N-acyl chain of 16 to 17 carbons in length and a hydroxyl group on the β-rather than the α-carbon and (2) lacking a 4-hydroxy group on its sphingoid base; optionally, (3) there can be an iso-branched lipid terminus Such chemical entities include but are not limited to the α-GalCer molecules produced by various bacterial species that live in the digestive tracts of animals, including humans, and hence are constituents of the gut microbiota (Thursby and Juge, 2017). Referred to collectively as “commensal bacteria” or “commensals,” a majority of these bacterial species have been found in the intestinal lumen, but many commensals also have adapted to colonize different anatomical locations in the intestine, including the surface of intestinal epithelial cells and the interior of gut-associated lymphoid tissues (Fung et al., 2014).

The term “endocytosis” encompasses (1) phagocytosis and (2) pinocytosis, itself a category inclusive of (2a) macropinocytosis, as well as of (2b) clathrin-mediated endocytosis, (2c) caveolae-mediated endocytosis and (2d) clathrin-/caveolae-independent endocytosis, all of which tend to access the late-endosome/lysosome pathway. Macropinocytosis can occur when multiple fixed location antibodies or ligands, for example as found on the surface of a bispecific antibody-targeted minicell, bind to multiple receptors on a mammalian cell surface, in effect disrupting mammalian receptor dimerization which normally occurs when such receptors are free to laterally diffuse within the mammalian cell plasma membrane. This disruption can result in the mammalian cell activating the process of forming pseudopodia to engulf the entire minicell, since receptor-mediated endocytosis generally cannot proceed when multiple ligands and receptors are clogged in the membrane, unable to dimerize. The interaction between the ligand on a minicell and a mammalian cell surface receptor, the present inventors discovered, activates micropinocytosis to the late-endosomal/lysosomal compartment. By virtue of such an endocytosis pathway, the present inventors further discovered that the minicells were able to release their payload into the cytoplasm of the target mammalian cell. In the event the payload is an encoding nucleic acid, some of the nucleic acid copies are degraded in the lysosome, but those that escape the lysosomal membrane may enter the mammalian cell cytoplasm for transport to the mammalian cell nucleus, where the encoded gene(s) are expressed.

The phrase “delivery element” as used herein, can refer to any vehicle by which a substance is delivered to a target. In particular, a delivery element is a vehicle into which a substance, e.g., a medicament, adjuvant, or other molecule, can be packaged, or a vehicle that can be associated with the substance, such that the delivery element facilitates the transport of the substance to a target, e.g., a target tissue, target cell, etc.

In the present context, the delivery element category includes, without limitation, intact mammalian cells and intact bacterial cells, either active (live) or inactivated (killed). An intact mammalian cell in this regard can be, for example, an immune cell, such as a natural killer cell, a dendritic cell, or a macrophage.

The category of delivery elements also includes vesicles derived from cells, such as intact minicells derived from bacterial cells (see below), and so-called extracellular vesicles or EVs derived from eukaryotic cells. EVs are lipid bilayer-delimited particles that are naturally released from almost all types of cells, mammalian and non-mammalian. Illustrative of EVs are exosomes, which originate from the endosomal system, and microvesicles, which are shed from plasma membrane. For a review of the molecular biology of EVs, see van Niel et al., 2018, which is incorporated herein by reference in its entirety.

Any conventional approach to bacterial fermentation, employing free or immobilized commensal bacterial cells in a stirred fermentation tank, can be readily adapted for producing the type of α-GalCer compounds used in the present invention, as described above. See generally Lang, 1999.

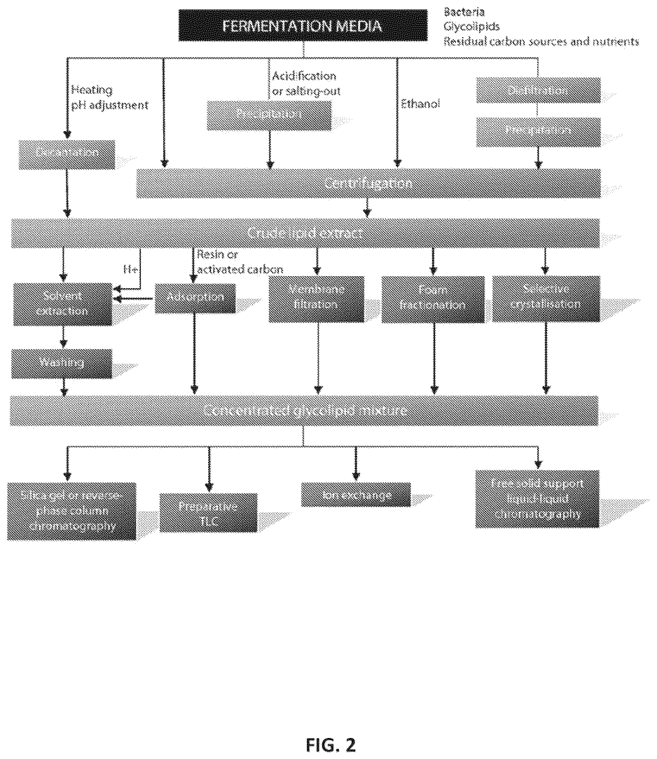

Purification of α-GalCer molecules from the bacterial cells produced from such bacterial fermentations can be accomplished via any of a variety of glycolipid-isolation methodologies employed in the field. In principle, the extraction of microbial glycolipids requires a preliminary separation of the microorganisms from the fermentation media (FIG. 2). At an industrial scale, bacteria are typically collected by centrifugation. A variation on this approach entails adding ethanol in order to dissolve the glycolipids and then removing the cells by centrifugation. Other methods based on precipitation may be used to remove bacterial α-GalCer molecules from fermentation media. For instance, acidification or salting-out of the media makes the glycolipids less soluble in the aqueous phases. After precipitation, they can be collected by centrifugation.

The aforementioned centrifugation and precipitation methods can be deployed conveniently when working with large volumes, but the resulting crude extracts may remain contaminated by lipids or carbohydrate compounds. Solvent extraction can be a more efficient alternative to isolate glycolipid mixtures more cleanly. In this regard, polar organic solvents such as chloroform, methanol, or ethyl acetate can be used. An additional extraction with apolar solvents such as n-hexane, pentane or methyl tert-butyl ether also can be useful to remove residual lipid substrates, which may be co-extracted with the bacterial α-GalCer molecules. The extraction yield can be improved, moreover, if a preliminary acidification step is performed to reduce solubility in water.

The present inventors discovered that preparative thin layer chromatography (PTLC) is an effective method for purification of bacterial α-GalCer molecules to pharmaceutical grade.

This technique has been used in purifying and characterizing an α-GalCer at the laboratory scale (Wieland et al., 2013) but never at pharmaceutical scale, as further described below.

In this description, “pharmaceutical grade” denotes a composition in which the active constituent-here, bacterial α-GalCer molecules-accounts for between about 97% and about 99% by weight, based on the total weight of all organic molecules of the composition.

In some embodiments, purification of bacterial α-GalCer molecules results in a bacterial α-GalCer sample of at least 95% purity, at least 96% purity, at least 97% purity, at least 98% purity, at least 99% purity, or 100% purity. In some embodiments, a purified bacterial α-GalCer sample is substantially free of non-bacterial α-GalCer components. As used here, “substantially free” can refer to a bacterial α-GalCer composition that contains less than 2% (e.g., less than 1.5%, less than 1%, less than 0.5%, less than 0.1%, or less than 0.01%) non-bacterial α-GalCer components. In some embodiments, a bacterial α-GalCer composition comprises more than 90% by weight, more than 91 % by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of a bacterial-GalCer compound. In some embodiments, purification of bacterial α-GalCer molecules results in a bacterial α-GalCer sample of between about 95% purity and about 99.9% purity. In some embodiments, purification of bacterial α-GalCer molecules results in a bacterial α-GalCer sample of between about 97% purity and about 99% purity. In some embodiments, a bacterial α-GalCer composition comprises between about 95% by weight and about 99.9% by weight of a bacterial α-GalCer compound. In some embodiments, a bacterial α-GalCer composition comprises between about 97% by weight and about 99% by weight of a bacterial α-GalCer compound.

By way of illustration, a B. fragilis strain is grown anaerobically in basal medium supplemented with hemin (50 μg/ml) and vitamin K1 (0.5μg/ml) at 37° C. for 2 days. The cells are harvested by centrifugation and extracted with CHCl₃:MeOH (2:1). The organic extract is filtered, concentrated, and then re-dissolved in CHCl₃:MeOH (2:1) and treated with 0.5 N NaOH. The suspension is stirred at 37° C. for 1 h, then brought to pH 2-4 with 10% HCl. The aqueous layer is extracted 3 times with CHCl₃:MeOH (2:1). The organic layers are combined, dried (Na₂SO₄), and concentrated to give a crude extract. The crude extract is dissolved in a minimum amount of CHCl₃:MeOH (2:1), applied to a 2 mm PTLC plate and eluted with CHCl₃:MeOH:AcOH:H₂O (100:20:12:5). The plate is divided into three sections based on polarity (top, middle, and bottom). Each section is scraped off the plate and extracted with CHCl₃:MeOH (5:1). The resulting solution is concentrated and further purified by preparative TLC (0.5 mm): the top section is eluted in CHCl₃:MeOH:NH₄OH (95:5:0.8) to give purified ceramidel (white solid, Rf=0.3). The middle section is eluted in CHCl₃:MeOH:H₂O (first in 88:12:0.5, then in 65:25:4) to give α-galactosylceramide (α-GalCer_{Bf}, glass, Rf=0.6 in 65:24:4). The bottom section is eluted in CHCl₃:MeOH:AcOH:H₂O (100:20:12:5) to give purified ceramide phosphorylethanolamine (white solid, Rf=0.2). Each compound is isolated as a mixture of compounds with varying lipid chain lengths and is not further separated. The α-GalCer compound, in this case α-GalCer_{Bf}, comprises ~0.5% of the extractable lipids, as determined by HPLC/MS.

The inventors determined that 1×10⁹ minicells can package about 100 ng of an α-GalCer compound in accordance with the present invention. By way of phase I/IIa clinical trials treating end-stage cancer patients suffering from a variety of different cancers, the inventors further discovered that about 1×10¹⁰ minicells packaged with an α-GalCer compound is generally a therapeutically effective dose, i.e., about 0.001 mg of compound, which in turn can be contained in single-dose or single-use vial for human use. In this regard, doses on the order of 10-fold greater than 1×10¹⁰ minicells, i.e., 1.1×10¹⁰ minicells, 1.2×10¹⁰ minicells, 1.3×10¹⁰ minicells, and so on to about 1×10¹¹ minicells, should prove safe and effective for various therapeutic indications, discussed below in more detail.

Informed by results from phase IIa clinical trials, the inventors determined that stabilization of such end-stage cancer patients could take as many as 50 doses per year of minicells packaged with an α-GalCer compound according to the present invention. Thus, 2 million of the aforementioned vials would be enough to treat approximately 40,000 patients, a number illustrative of a cancer indication treated globally (“pharmaceutical scale”).

For pharmaceutical scale manufacturing pursuant to the present invention, therefore, one would require approximately 2 gm of the α-GalCer compound for the minimum of 2 million vials. The PTLC purification methodology described above can provide, in a single run, about 1 gm of pharmaceutical-grade bacterial α-GalCer, sufficient for purposes of manufacturing at pharmaceutical scale of a medicament according to the present invention.

Among other aspects of the present invention, the paragraphs that follow describe loading of α-GalCer_{B} molecules into bacterially-derived minicells and killed bacterial cells, two types of delivery elements in accordance with the invention (see above). The approach to such loading entails co-incubating minicells and/or killed bacterial cells with the α-GalCer_{B} in a buffer.

It is an insight of the present inventors that essentially the same approach can be used to load α-GalCer_{B} molecules into other types of delivery elements, including intact mammalian cells and extracellular vesicles (EVs) such as exosomes. Thus, mammalian cells are known to be highly receptive to uptake of exogenously located peptides and nucleic acids, etc., and a glycolipid (here, α-GalCer_{B}) likewise would be expected, in a co-incubation context, to fuse readily with the hydrophobic membrane of a mammalian cell to effect loading of the cell.

Exemplary of EVs, moreover, exosomes are known to be loadable by co-incubation (see Kim et al., 2020), as well as by an alternative pre-loading strategy involving introduction into parental cells of exogenous active agent, which then is released into exosomes. See Gehrman et al., 2013 (exosomes loaded with α-GalCer are derived from α-GalCer-pulsed dendritic cells).

### II. Encapsulation of α-Galactosylceramide Compounds and Anti-Neoplastic Agents

The α-galactosylceramide compounds of the present disclosure, i.e., α-GalCer_{B}, can be effectively delivered to phagocytic cells by encapsulating the antigen using intact, bacterially-derived minicells or killed bacterial cells that can be taken up by macrophages and/or by dendritic cells.

In some embodiments, the α-GalCer_{B} is administered in combination with an antineoplastic agent that also is encapsulated, for example, using intact, bacterially derived minicells or killed bacterial cells. In some embodiments, the α-GalCer_{B} is administered in combination with an antineoplastic agent, where both the α-GalCer_{B} and the antineoplastic agent are encapsulated in intact, bacterially derived minicells or killed bacterial cells. In some embodiments, the α-GalCer_{B} and the antineoplastic agent are encapsulated in the same minicell or killed bacterial cell. In some embodiments, the α-GalCer_{B} and the antineoplastic agent are encapsulated in separate minicells or killed bacterial cells. In some embodiments, the encapsulated α-GalCer_{B} is administered with an antineoplastic agent that is not encapsulated.

### Intact Bacterially-Derived Minicells

The term “minicell” is used here to denote a derivative of a bacterial cell that lacks chromosomes (“chromosome-free”) and is engendered by a disturbance in the coordination, during binary fission, of cell division with DNA segregation. Minicells are distinct from other small vesicles, such as so-called “membrane blebs” (about 0.2 μm or less in size), which are generated and released spontaneously in certain situations but which are not due to specific genetic rearrangements or episomal gene expression. By the same token, intact minicells are distinct from bacterial ghosts, which are not generated due to specific genetic rearrangements or episomal gene expression. Bacterially derived minicells employed in this disclosure are fully intact and thus are distinguished from other chromosome-free forms of bacterial cellular derivatives characterized by an outer or defining membrane that is disrupted or degraded, even removed. See U.S. Pat. No. 7,183,105 at col. 111, lines 54 et seq. The intact membrane that characterizes the minicells of the present disclosure allows retention of the therapeutic payload within the minicell until the payload is released, post-uptake, within a phagocytic cell or tumor cell.

Minicells are anucleate, non-living nanoparticles produced as a result of inactivating the genes that control normal bacterial cell division, thereby de-repressing polar sites of cell. Ma et al., 2004. The de-repression means that the bacteria divide in the center as well as at the poles; the polar division resulting in minicells which the inventors of the present disclosure have shown can function as leak-resistant, micro-reservoir carriers that allow efficient packaging of a range of different chemotherapeutic drugs. Moreover, in contrast to current stealth liposomal drug carriers like DOXIL (liposomal doxorubicin), for example, that can package only ~14,000 molecules per particle (Park et al., 2002), or “armed antibodies,” which can carry fewer than 5 drug molecules, minicells can readily accommodate payloads of up to 1 million drug molecules. Further, minicells can be targeted to over-expressed receptors on the surface of cancer cells using bispecific antibodies, which allows highly significant tumor growth-inhibition and/or regression, both in vitro and in vivo.

The minicells employed in the present invention can be prepared from bacterial cells, such as E. coli and S. typhymurium. Prokaryotic chromosomal replication is linked to normal binary fission, which involves mid-cell septum formation. In E. coli, for example, mutation of min genes, such as minCD, can remove the inhibition of septum formation at the cell poles during cell division, resulting in production of a normal daughter cell and a chromosome-less minicell. See de Boer et al., 1992; Raskin & de Boer, 1999; Hu & Lutkenhaus, 1999; Harry, 2001.

In addition to min operon mutations, chromosome-less minicells also are generated following a range of other genetic rearrangements or mutations that affect septum formation, for example, in the divIVB 1 in B. subtilis (Reeve and Cornett, 1975). Minicells also can be formed following a perturbation in the levels of gene expression of proteins involved in cell division/chromosome segregation. For instance, over-expression of minE leads to polar division and production of minicells. Similarly, chromosome-less minicells can result from defects in chromosome segregation, e.g., the smc mutation in Bacillus subtilis (Britton et al., 1998), the spoOJ deletion in B. subtilis (Ireton et al., 1994), the mukB mutation in E. coli (Hiraga et al., 1989), and the parC mutation in E. coli (Stewart and D'Ari, 1992). Further, CafA can enhance the rate of cell division and/or inhibit chromosome partitioning after replication (Okada et al., 1994), resulting in formation of chained cells and chromosome-less minicells.

Accordingly, minicells can be prepared for the present disclosure from any bacterial cell, be it of Gram-positive or Gram-negative origin due to the conserved nature of bacterial cell division in these bacteria. Furthermore, the minicells used in the disclosure should possess intact cell walls (i.e., are “intact minicells”), as noted above, and should be distinguished over and separated from other small vesicles, such as membrane blebs, which are not attributable to specific genetic rearrangements or episomal gene expression.

In a given embodiment, the parental (source) bacteria for the minicells can be Gram positive, or they can be Gram negative. In one aspect, the parental bacteria are one or more selected from Terra-/Glidobacteria (BV1), Proteobacteria (BV2), BV4 including Spirochaetes, Sphingobacteria, and Planctobacteria. Pursuant to another aspect, the bacteria are one or more selected from Firmicutes (BV3) such as Bacilli, Clostridia or Tenericutes/Mollicutes, or Actinobacteria (BV5) such as Actinomycetales or Bifidobacteriales.

Pursuant to the invention, killed bacterial cells are non-living prokaryotic cells of bacteria, cyanobateria, eubacteria and archaebacteria, as defined in the 2nd edition of Bergey's Manual of Systematic Biology. Such cells are deemed to be “intact” if they possess an intact cell wall and/or cell membrane and contain genetic material (nucleic acid) that is endogenous to the bacterial species. Methods of preparing killed bacterial cells are described, for instance, in U.S. Pat. No. 9,878,043.

In yet a further aspect, the bacteria are one or more selected from Eobacteria (Chloroflexi, Deinococcus-Thermus), Cyanobacteria, Thermodesulfobacteria, thermophiles (Aquificae, Thermotogae), Alpha, Beta, Gamma (Enterobacteriaceae), Delta or Epsilon Proteobacteria, Spirochaetes, Fibrobacteres, Chlorobi/Bacteroidetes, Chlamydiae/Verrucomicrobia, Planctomycetes, Acidobacteria, Chrysiogenetes, Deferribacteres, Fusobacteria, Gemmatimonadetes, Nitrospirae, Synergistetes, Dictyoglomi, Lentisphaerae Bacillales, Bacillaceae, Listeriaceae, Staphylococcaceae, Lactobacillus, Enterococcaceae, Lactobacilluseae, Leuconostocaceae, Streptococcaceae, Clostridiales, Halanaerobiales, Thermoanaerobacterales, Mycoplasmatales, Entomoplasmatales, Anaeroplasmatales, Acholeplasmatales, Haloplasmatales, Actinomycineae, Actinomycetaceae, Corynebacterineae, Nocardiaceae, Corynebacteriaceae, Frankineae, Frankiaceae, Micrococcineae, Brevibacteriaceae, and Bifidobacteriaceae.

### Loading Active Agents Into Minicells and Killed Bacterial Cells

Active agents, such as an α-galactosylceramide compound (i.e., α-GalCer_{B}), or antineoplastic agents, such as small molecular drugs, proteins and functional nucleic acids, can be packaged into minicells directly by co-incubating a plurality of intact minicells with the active agent in a buffer. The buffer composition can be varied, as a function of conditions well known in this field, to optimize the loading of the active agent in the intact minicells. An exemplary buffer suitable for loading includes, but is not limited to, phosphate buffered saline (PBS). Once packaged, the active agent remains inside the minicell and is protected from degradation.

Active agents such as functional nucleic acids or proteins that can be encoded for by a nucleic acid, can be introduced into minicells by transforming into the parental bacterial cell a vector, such as a plasmid, that encodes the active agents. When a minicell is formed from the parental bacterial cell, the minicell retains certain copies of the plasmid and/or the expression product, the antineoplastic agent. More details of packaging and expression product into a minicell is provided in WO 2003/033519, the contents of which are incorporated into the present disclosure in its entirety by reference.

Data presented in WO 2003/033519 demonstrated, for example, that recombinant minicells carrying mammalian gene expression plasmids can be delivered to phagocytic cells and to non-phagocytic cells. WO 2003/033519 also described the genetic transformation of minicell-producing parent bacterial strains with heterologous nucleic acids carried on episomally-replicating plasmid DNAs. Upon separation of parent bacteria and minicells, some of the episomal DNA segregated into the minicells. The resulting recombinant minicells were readily engulfed by mammalian phagocytic cells and became degraded within intracellular phagolysosomes. Moreover, some of the recombinant DNA escaped the phagolysosomal membrane and was transported to the mammalian cell nucleus, where the recombinant genes were expressed.

In other embodiments, multiple nucleic acids directed to different mRNA targets can be packaged in the same minicell. Such an approach can be used to combat drug resistance and apoptosis resistance. For instance, cancer patients routinely exhibit resistance to chemotherapeutic drugs. Such resistance can be mediated by over-expression of genes such as multi-drug resistance (MDR) pumps and anti-apoptotic genes, among others. To combat this resistance, minicells can be packaged with therapeutically significant concentrations of functional nucleic acid to MDR-associated genes and administered to a patient before chemotherapy. Furthermore, packaging into the same minicell multiple functional nucleic acid directed to different mRNA targets can enhance therapeutic success since most molecular targets are subject to mutations and have multiple alleles. More details of directly packaging a nucleic acid into a minicell is provided in WO 2009/027830, the contents of which are incorporated into the present disclosure in its entirety by reference.

Small molecule drugs, whether hydrophilic or hydrophobic, can be packaged in minicells by creating a concentration gradient of the drug between an extracellular medium comprising minicells and the minicell cytoplasm. When the extracellular medium comprises a higher drug concentration than the minicell cytoplasm, the drug naturally moves down this concentration gradient, into the minicell cytoplasm. When the concentration gradient is reversed, however, the drug does not move out of the minicells. More details of the drug loading process and its surprising nature are found, for instance, in U.S. Pat. No. 8,772,013, the contents of which are specifically incorporated by reference.

To load minicells with drugs that normally are not water soluble, the drugs initially can be dissolved in an appropriate solvent. For example, paclitaxel can be dissolved in a 1:1 blend of ethanol and cremophore EL (polyethoxylated castor oil), followed by a dilution in PBS to achieve a solution of paclitaxel that is partly diluted in aqueous media and carries minimal amounts of the organic solvent to ensure that the drug remains in solution. Minicells can be incubated in this final medium for drug loading. Thus, the inventors discovered that even hydrophobic drugs can diffuse into the cytoplasm or the membrane of minicells to achieve a high and therapeutically significant cytoplasmic drug load. This is unexpected because the minicell membrane is composed of a hydrophobic phospholipid bilayer, which would be expected to prevent diffusion of hydrophobic molecules into the cytoplasm. The loading into minicells of a diversity of representative small molecule drugs has been shown, illustrating different sizes and chemical properties: doxorubicin, paclitaxel, fluoro-paclitaxel, cisplatin, vinblastine, monsatrol, thymidylate synthase (TS) inhibitor OSI-7904, irinotecan, 5-fluorouracil, gemcitabine, and carboplatin. Across the board, moreover, the resultant, small molecule drug-packaged minicells show significant anti-tumor efficacy, in vitro and in vivo.

### Targeting Minicells to Specific Mammalian Cells and Tumors

The inventors discovered that blood vessels around tumor cells display a loss of integrity; that is, the vessels have large fenestrations and are “leaky,” even in the blood brain barrier (BBB) environment. When cancer cells establish, they secrete substances that promote the formation of new blood vessels-a process called angiogenesis. These blood vessels grow quickly and, unlike normal blood vessels, they are leaky with “holes” (fenestrations) ranging from 50 nm to 1.2 μm (hyperpermeable vasculature). Drug delivery particles such as liposomes are currently believed to effect tumor-targeting by a passive process involving extravasation from the leaky vasculature that supports the tumor microenvironment. Hobbs et al., 1998. Although it has been shown that the abnormal tumor microenvironment is characterized by interstitial hypertension, and that this phenomenon may limit access of anti-cancer antibody therapeutics, this does not appear to be an absolute barrier as is exemplified by immunoliposomes (Nielsen et al, 2002) and antibody conjugated to Quantum Dots (Gao et al., 2004). This phenomenon also holds true for the minicell, which has the added advantage of carrying a specifically directed tumor antibody.

Following IV injection the minicell extravasates into the tumor microenvironment and this is followed by active targeting via cancer cell-surface receptor engagement and endocytosis. In contrast to conventional understanding, therefore, particles that are as large as minicells, i.e., much larger than the above-discussed consensus pore size limitations of the BBB, nevertheless are smaller than the fenestrations in the walls of the leaky blood vessel; hence, they can extravasate passively through these fenestrations and into the tumor microenvironment.

Upon entering the tumor microenvironment, minicells are able to trigger macropinocytosis or receptor-mediated internalization by the host tumor cells and to be taken up by them. Thus, a minicell that is packaged with an antineoplastic agent will release the agent into the cytoplasm of the tumor cell, killing it.

Pursuant to a further aspect of this disclosure, minicells or killed bacterial cells that contain an antineoplastic agent and/or an α-galactosylceramide compound (i.e., α-GalCer_{B}) can be directed to a target mammalian tumor cell via a ligand. In some embodiments the ligand is “bispecific.” That is, the ligand displays a specificity for both minicell and mammalian (tumor) cell components, such that it causes a given vesicle to bind to the target cell, whereby the latter engulfs the former. Use of bispecific ligands to target a minicell to a tumor cell is further described in WO 2005/056749 and WO 2005/079854, and use of bispecific ligands to target a killed bacterial cell to a tumor cell is further described in U.S. Pat. No. 8,591,862. Once such a ligand is attached to a vesicle, the unoccupied specificity (“monospecificity”) of the ligand pertains until it interacts with the target (tumor) mammalian cell. A number of tumor targeting ligands are known in the art (Hong et al., 2011; Hoelder et al., 2012; Galluzzi et al., 2013).

Several peptides, such as somatostatin (SST) peptide, vasoactive intestinal peptide (VIP), Arg-Gly-Asp (RGD) peptide, and bombesin/gastrin-releasing peptide (BBN/GRP), have been successfully characterized for tumor receptor imaging (De Jong et al., 2009; Tweedle, 2009;

Schottelius and Wester 2009; Igarashi et al., 2011; Laverman et al., 2012).

Tumor-targeting peptide sequences can be selected mainly in three different ways: (1) derivatization from natural proteins (Nagpal et al., 2011); (2) chemical synthesis and structure-based rational engineering (Andersson et al., 2000; Merrifield, 2006); and (3) screening of peptide libraries (Gray and Brown 2013). Among the methods, phage display technology is a conventional but most widely used method with many advantages such as ease of handling and large numbers of different peptides can be screened effectively (Deutscher, 2010).

Receptors that are overexpressed on tumor cells rather than on normal cells are excellent candidates for in vivo tumor imaging. To date, many tumor targeting peptides and their analogs have been identified as described below.

Arg-Gly-Asp (RGD) peptide—RGD specifically binds to integrin receptors (Ruoslahti, 1996). Integrins constitute two subunits (α and β subunits). The integrin family, especially αVβ3, is associated with tumor angiogenesis and metastasis. They are overexpressed on endothelial cells during angiogenesis, but barely detectable in most normal organs. Therefore, they are widely used for diagnostic imaging.

Bombesin (BBN)/gastrin-releasing Peptide (GRP)—Amphibian BBNS and Their related peptides consist of a family of neuropeptides exhibiting various physiological effects such as exocrine and endocrine secretions, thermoregulation, sucrose regulations as well as cell growth (Ohki-Hamazaki et al., 2005). The bombesin-like peptide receptors have 4-subtypes: the neuromedin B receptor, the bombesin 3 receptor, the GRP receptor, and the bombesin 4 receptor. These receptors are overexpressed in many tumors such as breast cancer, ovarian cancer and gastrointestinal stromal tumors.

Cholecystokinin (CCK)/gastrin peptide—CCK and gastrin are structurally and functionally similar peptides that exert a variety of physiological actions in the gastrointestinal tract as well as the central nervous system (Matsuno et al., 1997). Three types of receptors for CCK (CCK1, CCK2 and CCK2i4sv have been identified, which all belong to the superfamily of GPCRs. Among them, CCK2/gastrin receptors have been frequently found in human cancers such as stromal ovarian cancers and astrocytomas.

α-Melanocyte-stimulating hormone (α-MSH)—α-MSHs are linear tridecapeptides, mainly responsible for skin pigmentation regulation (Singh and Mukhopadhyay, 2014). α-MSHs and their analogs exhibit binding affinities to melanocortin-1 receptors (MC-1r) which are expressed in over 80% of human melanoma metastases, and thus, are widely used as vehicles for melanoma-targeted imaging and radiotherapy.

Neuropeptide Y (NPY)—NPY is a 36 amino acid peptide and belongs to the pancreatic polypeptide family (Tatemoto, 2004). NPY receptors are overexpressed in various tumors including neuroblastomas, sarcomas, and breast cancers.

Neutrotensin (NT)—NT is a 13 amino acid peptide, targeting NT receptor which has been identified in various tumors such as ductal pancreatic adenocarcinomas, small cell lung cancer, and medullary thyroid cancer (Tyler-McMahon et al., 2000). Therefore, it is an attractive candidate for cancer imaging.

Prostate Specific Membrane Antigen (PSMA)-Prostate cancer cells overexpress PSMA on the cell surface (Silver et al., 2007; Ghosh and Heston, 2004; Mhawech-Fauceglia et al., 2007; Santoni et al., 2014). There are several available radiopharmaceuticals that target PSMA including [⁶⁸Ga]Ga-PSMA-HBED-CC (also known as [⁶⁸Ga]Ga-PSMA-11 [PET]), a monoclonal antibody (mAb) [¹⁷⁷Lu]Lu/[⁹⁰Y]Y-J591 (therapy), [¹²³I]I-MIP-1072 (planar/SPECT), [¹³¹I]I-MIP-1095 (therapy), and the theranostic agents PSMA-I&T and DKFZ-PSMA-617 (PSMA-617), which are labeled with ⁶⁸Ga for PET or with ¹⁷⁷Lu for therapy.

Somatostatin (SST) peptide—SSTs are naturally occurring cyclopeptide hormones with either 14 or 28 amino acids (Weckbecker et al., 2003). They can inhibit the secretion of insulin, glucagon and some other hormones. Somatostatin receptors (SSTRs; five subtypes SSTR1-SSTR5) are overexpressed in many tumors including gliomas, neuroendocrine tumors and breast tumor. Neuroendocrine neoplasia (NEN) of the GEP system originates most frequently from the pancreas, jejunum, ileum, cecum, rectum, appendix, and colon. The common characteristic of all GEP-NEN is the compound features of endocrine and nerve cells. Well-differentiated NEN overexpresses somatostatin receptors (SSTRs), especially the SSTR-2 subtype.

Substance P—Substance P is an undecapeptide belonging to a family of neuropeptides known as tachykinins (Strand, 1999). Substance P is a specific endogenous ligand known for neurokinin 1 receptor (NK1R) which is found to be expressed on various cancer cells.

T140—T140 is a 14 amino acid peptide with one disulfide bridge and is an inverse agonist of chemokine receptor type 4 (CXCR4) (Burger et al., 2005). Its derivatives are widely used as CXCR4 imaging agents.

Tumor molecular targeted peptide 1 (TMTP1 )—TMTP1 is a 5-amino acid peptide that has been found to specifically bind to highly metastatic cancer cells, especially those from a typical liver micrometastasis (Yang et al., 2008).

Vasoactive intestinal peptide (VIP)—VIP is a neuropeptide with 28 amino acids (Igarashi et al., 2011). It promotes vasodilation, cell growth and proliferation. Its action is mainly controlled by two receptor subtypes (VPAC1 and VPAC2). A large amount of VIP receptors is expressed on many tumors including adenocarcinomas of the pancreas and neuroendocrine tumors.

The ligand can be attached to the cell membrane of the vesicles by virtue of the interaction between the ligand and a component on the cell membrane, such as a polysaccharide, a glycoprotein, or a polypeptide. The expressed ligand is anchored on the surface of a vesicle such that the tumor surface component-binding portion of the ligand is exposed so that the portion can bind the target mammalian cell surface receptor when the vesicle and the mammalian tumor cell come into contact.

Alternatively, the ligand can be expressed and displayed by a living counterpart of a bacterially derived vesicle, e.g., by the parent cell of a minicell or by a bacterial cell before it becomes a killed cell. In this instance the ligand does not require a specificity to the vesicle and only displays a specificity to a component that is characteristic of mammalian cells. That is, such component need not be unique to tumor cells, per se, or even to the particular kind of tumor cells under treatment, so long as the tumor cells present the component on their surface.

Upon intravenous administration, vesicles accumulate rapidly in the tumor microenvironment. This accumulation, occurring as a function of the above-described leaky tumor vasculature, effects delivery of vesicle-packaged therapeutic payload to cells of the tumor, which then internalize packaged vesicles.

The inventors have found that this delivery approach is applicable to a range of mammalian tumor cells, including cells that normally are refractory to specific adhesion and endocytosis of minicells. For instance, ligands that comprise an antibody directed at an anti-HER2 receptor or anti-EGF receptor can bind minicells to the respective receptors on a range of targeted non-phagocytic cells, such as lung, ovarian, brain, breast, prostate, and skin cancer cells.

The binding thus achieved precedes uptake of the vesicles by each type of non-phagocytic cells. That is, in the context of the present invention a suitable target cell presents a cell surface receptor the binding of which, by a ligand on a vesicle, elicits endocytosis of that vesicle.

More specifically, the present inventors discovered that the interaction between (a) the ligand on a minicell or a killed bacterial cell and (b) a mammalian cell surface receptor can activate an uptake pathway, called here a “receptor-mediated endocytosis” (rME) pathway, into the late-endosomal/lysosomal compartment of the target host cell, such as a tumor cell. By this rME pathway, the inventors found, bacterially derived vesicles are processed through the early endosome, the late endosome and the lysosome, resulting in release of their payload into the cytoplasm of the mammalian host cell. Moreover, a payload that is a nucleic acid not only escapes complete degradation in the late-endosomal/lysosomal compartment but also is expressed by the host cell.

A tumor targeting ligand for this delivery approach can be “bispecific,” as described above, because it binds to surface components on a payload-carrying vesicle and on a target cell, respectively, and its interaction with the latter component leads to uptake of the vesicle into the rME pathway. In any event, a given target cell surface receptor can be a candidate for binding by the ligand, pursuant to the invention, if interaction with the component in effect accesses an endocytic pathway that entails a cytosolic internalization from the target cell surface. Such candidates are readily assessed for suitability in the invention via an assay in which a cell type that presents on its surface a candidate component is co-incubated in vitro with minicells carrying a ligand that binds the candidate and that also is joined to a fluorescent dye or other marker amenable to detection, e.g., visually via confocal microscopy. (An in vitro assay of this sort is described by MacDiarmid et al., 2007b, in the legend to Figure 3 at page 436.) Thus, an observed internalization of the marker constitutes a positive indication by such an assay that the tested target cell surface receptor is suitable for the present invention.

In accordance with the invention, the ligand can be any polypeptide or polysaccharide that exhibits the desired specificity or specificities. Preferred ligands are antibodies. In its present use the term “antibody” encompasses an immunoglobulin molecule obtained by in vitro or in vivo generation of an immunogenic response. Accordingly, the “antibody” category includes monoclonal antibodies and humanized antibodies, such as single-chain antibody fragments (scFv), bispecific antibodies, etc. A large number of different bispecific protein and antibody-based ligands are known, as evidenced by the review article of Caravella and Lugovskoy, 2010. Antibodies useful in accordance with the present disclosure can be obtained by known recombinant DNA techniques.

By way of non-limiting example, therefore, an antibody that carries specificity for a surface component, such as a tumor antigen, can be used to target minicells to cells in a tumor to be treated. Illustrative cell surface receptors in this regard include any of the RTKs epidermal growth factor receptor (EGFR), vascular endothelial growth factor receptor (VEGFR), platelet-derived growth factor receptor (PDGFR) and insulin-like growth factor receptor (IGFR), each of which is highly expressed in several solid tumors, including brain tumors, and folate receptor, which is overexpressed in some pituitary adenomas. Such a bispecific ligand can be targeted as well to mutant or variant receptors, e.g., the IL-13Rα2 receptor, which is expressed in 50% to 80% of human glioblastoma multiforme tumors, see Wykosky et al., 2008; Jarboe et al., 2007; Debinski et al., 2000; and Okada et al., 1994), but which differs from its physiological counterpart IL4R/IL13R, expressed in normal tissues. See Hershey, 2003. Thus, IL13Rα2 is virtually absent from normal brain cells. See Debinski and Gibo, 2000. Additionally, tumors that metastasize to the brain may overexpress certain receptors, which also can be suitable targets. For instance, Da Silva et al., 2010, showed that brain metastases of breast cancer expressed all members of the HER family of RTKs. HER2 was amplified and overexpressed in 20% of brain metastases, EGFR was overexpressed in 21% of brain metastases, HER3 was overexpressed in 60% of brain metastases and HER 4 was overexpressed in 22% of brain metastases. Interestingly, HER3 expression was increased in breast cancer cells residing in the brain.

Illustrative of candidate target cell surface receptors are members of the receptor tyrosine kinases or “RKTs,” a family of transmembrane proteins that undergo constitutive internalization (endocytosis) at a rate similar to that of other integral membrane proteins. See Goh and Sorkin, 2013. The family of RKTs is described by Lemmon and Schlessinger, Cell, 141(7): 1117-134 (2010). Exemplary RTKs are ErbB EGFR, ErbB2, ErbB3, ErbB4 Ins InsR, IGF1R, InsRR PDGF PDGFRα, PDGFRβ, CSF1R/Fms, Kit/SCFR, Fit3/Flk2 VEGF VEGFR1/Fit1, VEGFR2/KDR, VEGFR3/Fit4 FGF FGFR1, FGFR2, FGFR3, FGFR4 PTK7 PTK7/CCK4 Trk TrkA, TrkB, TrkC Ror Ror1, Ror2 MuSK Met, Ron Axl, Mer, Tyro3 Tie Tie1, Tie2 Eph EphA1-8, EphA10, EphB1-4, EphB6 Ret Ryk DDR DDR1, DDR2 Ros LMR LMR1, LMR2, LMR3 ALK, LTK STYK1 SuRTK106/STYK1.

Another candidate for suitable target cell surface receptors are the family of membrane-associated, high-affinity folate binding proteins (folate receptor), which bind folate and reduced folic acid derivatives and which mediate delivery of tetrahydrofolate to the interior of cells; the family of membrane-bound cytokine receptors that play a role in the internalization of a cognate cytokine, such as IL13; the surface antigens such as CD20, CD33, mesothelin and HM1.24, that are expressed on certain cancer cells and that mediate the internalization of cognate monoclonal antibodies, e.g., rituximab in the instance of CD20; and the family of adhesion receptors (integrins), which are transmembrane glycoproteins that are trafficked through the endosomal pathway and are major mediators of cancer cell adhesion. In one embodiment of the invention, the tumor cell surface receptor comprises an integrin, neuromedin B receptor, bombesin 3 receptor, GRP receptor, bombesin 4 receptor, CCK2/gastrin, melanocortin-1 receptor (MC-1r), neuropeptide Y (NPY) receptor, neutrotensin (NT) receptor, prostate specific membrane antigen (PSMA), somatostatin (SST) receptor, neurokinin 1 receptor (NK1R), chemokine receptor type 4 (CXCR4), vasoactive intestinal peptide (VIP), epidermal growth factor receptor (EGFR), vascular endothelial growth factor receptor (VEGFR), platelet-derived growth factor receptor (PDGFR), insulin-like growth factor receptor (IGFR), or any combination thereof.

According to another embodiment of the invention, the cell surface receptor is an antigen which is uniquely expressed on a target cell in a disease condition, but which remains either non-expressed, expressed at a low level or non-accessible in a healthy condition. Examples of such target antigens which might be specifically bound by a targeting ligand of the invention may advantageously be selected from EpCAM, CCR5, CD19, HER-2 neu, HER-3, HER-4, EGFR, PSMA, CEA, MUC-1 (mucin), MUC2, MUC3, MUC4, MUC5, MUC5, MUC7, BhcG, Lewis-Y. CD20, CD33, CD30, ganglioside GD3, 9-O-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRVIII, LG, SAS and CD63. Receptors that are found on specific types of tumor cells and that could be targeted by the bispecific ligand include, but are not limited to: disialoganglioside GD2, which is overexpressed in, inter alia, neuroblastoma, small cell lung cancer, Ewing sarcoma, and osteosarcoma; CD19, which is overexpressed in most acute lymphoblastic leukemias (ALL), chronic lymphocytic leukemias (CLL), and B cell lymphomas; CD20, which is expressed in a majority of B-cell malignancies, including chronic lymphocytic leukemia, diffuse large B-cell lymphoma, follicular lymphoma, and mantle cell lymphoma; asialoglycoprotein, which is overexpressed in liver and colon cancers; mesothelin, which is overexpressed in mesothelioma; DLL3, which is highly expressed in SCLC and other neuroendocrine tumors; PSMA, which is highly expressed in prostate cancer cells as high as 100 to 1,000 times compared with in normal prostate cells, and even higher in advanced stages and castration-resistant prostate cancers (CRPCs); and CA-125, which is highly expressed in Epithelial ovarian carcinoma (including fallopian tube and primary serous peritoneal carcinoma), Endometrial carcinoma, and Endocervical adenocarcinoma.

### Purity

Minicells of the invention are substantially free from contaminating parent bacterial cells. Thus, minicell-comprising formulations preferably comprise fewer than about 1 contaminating parent bacterial cell per 10⁷ minicells, fewer than about 1 contaminating parent bacterial cell per 10⁸ minicells, fewer than about 1 contaminating parent bacterial cell per 10⁹ minicells, fewer than about 1 contaminating parent bacterial cell per 10¹⁰ minicells, or fewer than about 1 contaminating parent bacterial cell per 10¹¹ minicells.

Methods of purifying minicells are known in the art and described in PCT/IB02/04632. One such method combines cross-flow filtration (feed flow is parallel to a membrane surface; Forbes, 1987) and dead-end filtration (feed flow is perpendicular to the membrane surface). Optionally, the filtration combination can be preceded by a differential centrifugation, at low centrifugal force, to remove some portion of the bacterial cells and thereby enrich the supernatant for minicells.

Another purification method employs density gradient centrifugation in a biologically compatible medium. After centrifugation, a minicell band is collected from the gradient, and, optionally, the minicells are subjected to further rounds of density gradient centrifugation to maximize purity. The method may further include a preliminary step of performing differential centrifugation on the minicell-containing sample. When performed at low centrifugal force, differential centrifugation will remove some portion of parent bacterial cells, thereby enriching the supernatant for minicells.

Particularly effective purification methods exploit bacterial filamentation to increase minicell purity. Thus, a minicell purification method can include the steps of (a) subjecting a sample containing minicells to a condition that induces parent bacterial cells to adopt a filamentous form, followed by (b) filtering the sample to obtain a purified minicell preparation.

Known minicell purification methods also can be combined. One highly effective exemplary combination of methods is as follows:
Step A: Differential centrifugation of a minicell producing bacterial cell culture. This step, which may be performed at 2,000 g for about 20 minutes, removes most parent bacterial cells, while leaving minicells in the supernatant;
Step B: Density gradient centrifugation using an isotonic and non-toxic density gradient medium. This step separates minicells from many contaminants, including parent bacterial cells, with minimal loss of minicells. Preferably, this step is repeated within a purification method;
Step C: Cross-flow filtration through a 0.45 μm filter to further reduce parent bacterial cell contamination.
Step D: Stress-induced filamentation of residual parent bacterial cells. This may be accomplished by subjecting the minicell suspension to any of several stress-inducing environmental conditions;
Step E: Antibiotic treatment to kill parent bacterial cells;
Step F: Cross-flow filtration to remove small contaminants, such as membrane blebs, membrane fragments, bacterial debris, nucleic acids, media components and so forth, and to concentrate the minicells. A 0.2 μm filter may be employed to separate minicells from small contaminants including free lipopolysaccharide, and a 0.1 μm filter may be employed to concentrate minicells;
Step G: Dead-end filtration to eliminate filamentous dead bacterial cells. A 0.45 μm filter may be employed for this step; and
Step H: Removal of endotoxin from the minicell preparation. Anti-Lipid A coated magnetic beads may be employed for this step.

For pharmaceutical use, a composition of the disclosure should comprise minicells or killed bacterial cells that are isolated as thoroughly as possible from immunogenic components and other toxic contaminants. Methodology for purifying bacterially derived minicells to remove free endotoxin and parent bacterial cells are described, for example, in WO 2004/113507. Briefly, the purification process achieves removal of (a) smaller vesicles, such as membrane blebs, which are generally smaller than 0.2 μm in size, (b) free endotoxins released from cell membranes, and (c) parental bacteria, whether live or dead, and their debris, which also are sources of free endotoxins. Such removal can be implemented with, inter alia, a 0.2 μm filter to remove smaller vesicles and cell debris, a 0.45 μm filter to remove parental cells following induction of the parental cells to form filaments, antibiotics to kill live bacterial cells, and antibodies against free endotoxins.

Underlying the purification procedure is a discovery by the present inventors that, despite the difference of their bacterial sources, all intact minicells are approximately 400 nm in size, i.e., larger than membrane blebs and other smaller vesicles and yet smaller than parental bacteria. Size determination for minicells can be accomplished by using solid-state, such as electron microscopy, or by liquid-based techniques, e.g., dynamic light scattering. The size value yielded by each such technique can have an error range, and the values can differ somewhat between techniques. Thus, the size of minicells in a dried state can be measured via electron microscopy as approximately 400 nm±50 nm. Dynamic light scattering can measure the same minicells to be approximately 500 nm±50 nm in size. Also, drug-packaged, ligand-targeted minicells can be measured, again using dynamic light scattering, to be approximately 400 nm to 600 nm±50 nm.

This scatter of size values is readily accommodated in practice, e.g., for purposes of isolating minicells from immunogenic components and other toxic contaminants, as described above. That is, an intact, bacterially derived minicell is characterized by cytoplasm surrounded by a rigid membrane, which gives the minicell a rigid, spherical structure. This structure is evident in transmission-electron micrographs, in which minicell diameter is measured, across the minicell, between the outer limits of the rigid membrane. This measurement provides the above-mentioned size value of 400 nm±50 nm.

Another structural element of a killed bacterial cells or a minicell derived from Gram-negative bacteria is the O-polysaccharide component of lipopolysaccharide (LPS), which is embedded in the outer membrane via the lipid A anchor. The component is a chain of repeat carbohydrate-residue units, with as many as 70 to 100 repeat units of four to five sugars per repeat unit of the chain. Because these chains are not rigid, in a liquid environment, as in vivo, they can adopt a waving, flexible structure that gives the general appearance of seaweed in a coral sea environment; i.e., the chains move with the liquid while remaining anchored to the minicell membrane.

Influenced by the O-polysaccharide component, dynamic light scattering can provide a value for minicell size of about 500 nm to about 600 nm, as noted above. Nevertheless, minicells from Gram-negative and Gram-positive bacteria alike readily pass through a 0.45 um filter, which substantiates an effective minicell size of 400 nm ±50 nm. The above-mentioned scatter in sizes is encompassed by the present invention and, in particular, is denoted by the qualifier “approximately” in the phrase “approximately 400 nm in size” and the like.

In relation to toxic contaminants, a composition of the disclosure preferably comprises less than about 350 EU free endotoxin. Illustrative in this regard are levels of free endotoxin of about 250 EU or less, about 200 EU or less, about 150 EU or less, about 100 EU or less, about 90 EU or less, about 80 EU or less, about 70 EU or less, about 60 EU or less, about 50 EU or less, about 40 EU or less, about 30 EU or less, about 20 EU or less, about 15 EU or less, about 10 EU or less, about 9 EU or less, about 8 EU or less, about 7 EU or less, about 6 EU or less, about 5 EU or less, about 4 EU or less, about 3 EU or less, about 2 EU or less, about 1 EU or less, about 0.9 EU or less, about 0.8 EU or less, about 0.7 EU or less, about 0.6 EU or less, about 0.5 EU or less, about 0.4 EU or less, about 0.3 EU or less, about 0.2 EU or less, about 0.1 EU or less, about 0.05 EU or less, or about 0.01 EU or less.

A composition of the disclosure also can comprise at least about 10⁹ minicells or killed bacterial cells, e.g., at least about 1×10⁹, at least about 2×10⁹, at least about 5×10⁹, or at least 8×10⁹. In some embodiments, the composition comprises no more than about 10¹¹ minicells or killed bacterial cells, e.g., no more than about 1×10¹¹ or no more than about 9×10¹⁰, or no more than about 8×10¹⁰.

### III. Formulations

The invention includes within its scope compositions or formulations comprising intact, bacterially derived minicells or killed bacterial cells that comprise an α-galactosylceramide compound, such as an α-galactosylceramide compound that (1) comprises (i) a N-acyl chain of 16 to 17 carbons in length and (ii) a hydroxyl group on the β-rather than the α-carbon and (2) does not present a 4-hydroxy group on its sphingoid base (i.e., α-GalCer_{B}). In some embodiments, the formulations comprise bacterially-derived minicells or killed bacterial cells comprising an α-galactosylceramide compound (i.e., α-GalCer_{B}) alone or in combination with a carrier. In some embodiments, the formulations comprise bacterially-derived minicells or killed bacterial cells comprising an α-galactosylceramide compound (i.e., α-GalCer_{B}) alone, with a carrier, or in combination with an antineoplastic agent and a carrier. In some embodiments, the formulations comprise intact bacterially-derived minicells or killed bacterial cells comprising an an α-galactosylceramide compound (i.e., α-GalCer_{B}) and bacterially-derived minicells or killed bacterial cells comprising an antineoplastic agent. For example: (a) the α-GalCer_{B} and the antineoplastic agent can be comprised within the same minicell or killed bacterial cell; or (b) the α-GalCer_{B} can be comprised within a first minicell or killed bacterial cell, and the antineoplastic can be comprised within a second minicell or killed bacterial cell.

In an exemplary embodiment, the compositions disclosed herein comprise the α-glactosylceramide compound (i.e., α-GalCer_{B}) and an antineoplastic agent, wherein the α-glactosylceramide compound and the antineoplastic agent are comprised within one or more intact bacterially-derived minicells. In an exemplary embodiment, the compositions disclosed herein comprise the α-GalCer_{B} and the antineoplastic agent doxurubicin, wherein the α-GalCer_{B} and the doxurubicin are comprised within one or more intact bacterially-derived minicells.

In some embodiments, the formulations also optionally comprise at least one bispecific ligand for targeting the minicell to a target cell. The minicell and ligand may be any of those described herein. Thus, the bispecific ligand of the present invention is capable of binding to a surface component of the intact bacterially-derived minicell and to a surface component of a target mammalian cell.

A formulation comprising minicells, or killed bacterial cells, drugs (e.g., at least one antineoplastic agent) and optionally bispecific ligands of the present invention (that is, a formulation that includes such minicells, or killed bacterial cells, drugs and ligands with other constituents that do not interfere unduly with the drug or drug-delivering quality of the composition) can be formulated in conventional manner, using one or more pharmaceutically acceptable carriers or excipients.

Formulations or compositions of this disclosure can be presented in unit dosage form, e.g., in ampules or vials, or in multi-dose containers, with or without an added preservative. The formulation can be a solution, a suspension, or an emulsion in oily or aqueous vehicles, and can contain formulatory agents, such as suspending, stabilizing and/or dispersing agents. A suitable solution is isotonic with the blood of the recipient and is illustrated by saline, Ringer's solution, dextrose solution, or sterile water for injection. Alternatively, formulations can be in lyophilized powder form, for reconstitution with a suitable vehicle, e.g., sterile, pyrogen-free water or physiological saline. The formulations also can be in the form of a depot preparation. Such long-acting formulations can be administered by implantation (for instance, subcutaneously or intramuscularly) or by intramuscular injection. In some embodiments, administering comprises enteral or parenteral administration. In some embodiments administering comprises administration selected from oral, buccal, sublingual, intranasal, rectal, vaginal, intravenous, intramuscular, and subcutaneous injection.

In some aspects, a composition comprising an immunogenically effective amount of an α-galactosylceramide compound that (1) comprises (i) a N-acyl chain of 16 to 17 carbons in length and (ii) a hydroxyl group on the β-rather than the α-carbon and (2) does not present a 4-hydroxy group on its sphingoid base (i.e., α-GalCer_{B}) is provided. An “immunogenically effective amount” as used herein refers to the amount of α-GalCer_{B} sufficient to elicit an immune response. In the context of an α-GalCer_{B}, an immunogenically effective amount is the amount of antigen sufficient activate an iNKT cell response. The effectiveness of α-GalCer_{B} as an immunogen, can be assessed, for example, by measuring increases in production of cytokine, such as IFNγ, following administration.

In some aspects, a composition that includes a therapeutically effective amount of an antineoplastic agent is provided. A “therapeutically effective” amount of an anti-neoplastic agent is a dosage of the agent in question, e.g., a siRNA or a super-cytotoxic drug that invokes a pharmacological response when administered to a subject, in accordance with the present disclosure.

In the context of the present disclosure, therefore, a therapeutically effective amount can be gauged by reference to the prevention or amelioration of the tumor or a symptom of tumor, either in an animal model or in a human subject, when bacterially derived minicells or killed bacterial cells carrying a therapeutic payload are administered, as further described below. An amount that proves “therapeutically effective amount” in a given instance, for a particular subject, may not be effective for 100% of subjects similarly treated for the tumor, even though such dosage is deemed a “therapeutically effective amount” by skilled practitioners. The appropriate dosage in this regard also will vary as a function, for example, of the type, stage, and severity of the tumor.

When “therapeutically effective” is used to refer to the number of minicells or killed bacterial cells in a pharmaceutical composition, the number can be ascertained based on what antineoplastic agent is packaged into the minicells or killed bacterial cells and the efficacy of that agent in treating a tumor. The therapeutic effect, in this regard, can be measured with a clinical or pathological parameter such as tumor mass. A reduction or reduced increase of tumor mass, accordingly, can be used to measure therapeutic effects.

### IV. Administration Routes

Formulations of the invention can be administered via various routes and to various sites in a mammalian body, to achieve the therapeutic effect(s) desired, either locally or systemically. Delivery may be accomplished, for example, by oral administration, by application of the formulation to a body cavity, by inhalation or insufflation, or by parenteral, intramuscular, intravenous, intraportal, intrahepatic, peritoneal, subcutaneous, intratumoral, or intradermal administration. The α-GalCer_{B} and the antineoplastic agents can be administered by the same route or by different routes of administration. For example, the α-GalCer_{B} can be administered systemically and the antineoplastic agent can be administered locally. In some embodiments, both the α-GalCer_{B} and the antineoplastic agent are administered systemically.

The mode and site of administration is dependent on the location of the target cells. For example, the target phagocytic cells that uptake the α-GalCer_{B} can be found both in the tumor microenvironment and the in the vasculature associated with liver spleen and lymph nodes.

Accordingly, the α-GalCer_{B} may be delivered via targeted and/or non-targeted bacterially derived minicells or killed bacterial cells.

The antineoplastic agents can also be administered via targeted and/or non-targeted methods. For example, a tumor metastasis may be more efficiently treated via intravenous or intraperitoneal delivery of targeted compositions, such as, for example, intravenous or intraperitoneal delivery of targeted bacterially derived minicells. A combination of routes may also may be employed. For example, cytotoxic drug-loaded and receptor-targeted minicells may be administered locally as well as intravenously, and the α-GalCer_{B} minicells may be administered intravenously. The administration of targeted, drug-packaged minicells may target surface-exposed tumors, while the full combination of minicells administered intravenously may target tissue-localized tumors and also elicit the anti-tumor immune response.

### V. Administration Schedules

In general, the formulations disclosed herein may be used at appropriate dosages defined by routine testing, to obtain optimal physiological effect, while minimizing any potential toxicity. The dosage regimen may be selected in accordance with a variety of factors including age, weight, sex, medical condition of the patient; the severity of the condition to be treated, the route of administration, and the renal and hepatic function of the patient.

Optimal precision in achieving concentrations of α-GalCer_{B} and drug within the range that yields maximum efficacy with minimal side effects may require a regimen based on the kinetics of the α-GalCer_{B} and antineoplastic drug availability to target sites and target cells.

Distribution, equilibrium, and elimination of the α-GalCer_{B} and antineoplastic drug may be considered when determining the optimal concentration for a treatment regimen. The dosages of the α-GalCer_{B} and antineoplastic drugs may be adjusted when used in combination, to achieve desired effects.

Moreover, the dosage administration of the formulations may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See, e.g., WO 00/67776.

Specifically, the formulations of α-GalCer_{B} and/or antineoplastic drug may be administered at least once a week over the course of several weeks. In one embodiment, the formulations of α-GalCer_{B} and/or antineoplastic drug are administered at least once a week over several weeks to several months. The α-GalCer_{B} and one or more antineoplastic drugs can be administered simultaneously, sequentially, or intermittently in defined intervals.

More specifically, the formulations of α-GalCer_{B} and/or antineoplastic drug may be administered at least once a day for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or about 31 days. Alternatively, the formulations of α-GalCer_{B} and/or antineoplastic drug may be administered about once every day, about once every about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or about 31 days or more.

The formulations of α-GalCer_{B} and/or antineoplastic drug may alternatively be administered about once every week, about once every about 2, about 3, about 4, about 5, about 6, about 7, about 8, about 9, about 10, about 11, about 12, about 13, about 14, about 15, about 16, about 17, about 18, about 19 or about 20 weeks or more. Alternatively, the formulations of α-GalCer_{B} and/or antineoplastic drug may be administered at least once a week for about 2, about 3, about 4, about 5, about 6, about 7, about 8, about 9, about 10, about 11, about 12, about 13, about 14, about 15, about 16, about 17, about 18, about 19 or about 20 weeks or more.

The formulations of α-GalCer_{B} and/or antineoplastic drug may alternatively be administered about twice every week, about twice every about 2, about 3, about 4, about 5, about 6, about 7, about 8, about 9, about 10, about 11, about 12, about 13, about 14, about 15, about 16, about 17, about 18, about 19 or about 20 weeks or more. Alternatively, the formulations of α-GalCer_{B} and/or antineoplastic drug may be administered at least once a week for about 2, about 3, about 4, about 5, about 6, about 7, about 8, about 9, about 10, about 11, about 12, about 13, about 14, about 15, about 16, about 17, about 18, about 19 or about 20 weeks or more.

Alternatively, the formulations of α-GalCer_{B} and/or antineoplastic drug may be administered about once every month, about once every about 2, about 3, about 4, about 5, about 6, about 7, about 8, about 9, about 10, about 11 or about 12 months or more.

The formulations may be administered in a single daily dose, or the total daily dosage may be administered in divided doses of two, three, or four times daily.

In a method in which α-GalCer_{B} is administered after administration of an antineoplastic agent, administration of the antineoplastic agent may occur anytime from several minutes to several hours after administration of the α-GalCer_{B}. The antineoplastic agent may alternatively be administered anytime from several hours to several days, possibly several weeks up to several months after the α-GalCer_{B}.

More specifically, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23 or about 24 hours after the antineoplastic agent. Moreover, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or about 31 days after the administration of the antineoplastic agent. In yet another embodiment, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19 or about 20 weeks or more after the antineoplastic agent. In a further embodiment, the α-GalCer_{B} may be administered at least about 1, about 2, about 3, about 4, about 5, about 6, about 7, about 8, about 9, about 10, about 11 or about 12 months after the antineoplastic agent.

In a method in which α-GalCer_{B} is administered before administration of an antineoplastic agent, administration of the antineoplastic agent may occur anytime from several minutes to several hours before administration of the α-GalCer_{B}. The antineoplastic agent may alternatively be administered anytime from several hours to several days, possibly several weeks up to several months before the α-GalCer_{B}.

More specifically, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23 or about 24 hours before the antineoplastic agent. Moreover, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or about 31 days before the administration of the antineoplastic agent. In yet another embodiment, the α-GalCer_{B} may be administered at least about 1, about 2, about 3, about 4, about 5, about 6, about 7, about 8, about 9, about 10, about 11, about 12, about 13, about 14, about 15, about 16, about 17, about 18, about 19 or about 20 weeks or more before the antineoplastic agent. In a further embodiment, the α-GalCer_{B} may be administered at least about 1, about 2, about 3, about 4, about 5, about 6, about 7, about 8, about 9, about 10, about 11 or about 12 months before the antineoplastic agent.

### Methods of Treating Cancer

The compositions described herein may be used to treat a subject suffering from a cancer. The method disclosed herein comprises administering to the subject an immunogenically effective amount of a composition comprising intact, bacterially derived minicells or killed bacterial cells that encapsulate α-GalCer_{B} and an antineoplastic agent or therapy. In some embodiments, the α-GalCer_{B} is comprised in intact bacterially-derived minicells. In some embodiments, the α-GalCer_{B} and the antineoplastic agents are comprised in one or more intact bacterially-derived minicells. In some embodiments, the α-GalCer_{B} and the antineoplastic agents are comprised in separate intact bacterially-derived minicells. In some embodiments, the α-GalCer_{B} and the antineoplastic agents are comprised in the same intact bacterially-derived minicell. In some embodiments, bacterially derived minicells or killed bacterial cells that encapsulate α-GalCer_{B} are administered separately from the antineoplastic agent or therapy. In some embodiments, the α-GalCer_{B} and the antineoplastic agents are comprised in the same intact bacterially-derived minicell. In some embodiments, bacterially derived minicells or killed bacterial cells that encapsulate α-GalCer_{B} are administered simultaneously with the antineoplastic agent or therapy. In some embodiments, bacterially derived minicells or killed bacterial cells that encapsulate α-GalCer_{B} are administered in the same composition with the antineoplastic agent. In some embodiments, the intact, bacterially derived minicells or killed bacterial cells that encapsulate α-GalCer_{B} are administered as separate compositions with the antineoplastic agent. In another aspect, the compositions comprising the α-GalCer_{B} or antineoplastic agent used to treat a subject suffering from cancer further comprises a pharmaceutically acceptable carrier.

In another aspect, the methods disclosed herein are useful for treating a subject suffering from a cancer, wherein the subject is a human, a non-human primate, a dog, a cat, a cow, a sheep, a horse, a rabbit, a mouse, or a rat.

In another aspect, the methods disclosed herein are useful for treating a cancer disease. In some embodiment the cancer comprises a lung cancer, a breast cancer, a brain cancer, a liver cancer, a colon cancer, a pancreatic cancer, or a bladder cancer.

In some embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström's macroglobulinemia; or Wilms' tumor.

In some embodiments, the brain cancer or tumor is selected from the group consisting of brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma.

### VI. Definitions

A number of other phrases and terms, which are used in this description, are defined as follows.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to +4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to +4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that the component, range, dose form, etc. is suitable for the disclosed purpose.

In addition, amounts, ratios, and other numerical values are sometimes presented herein in a range format. Such range format is used for convenience and brevity and should be understood flexibly to include numerical values explicitly specified as limits of a range, but also to include all individual numerical values or sub-ranges encompassed within that range as if each numerical value and sub-range is explicitly specified. For instance, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In this description, “and/or” refers to and encompasses any and all possible combinations of one or more of the associated listed items, as well as the lack of combinations when interpreted in the alternative (“or”).

The term “comprising” is used herein to mean that the compositions and methods include the recited elements but not exclude others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

“Individual,” “subject,” “host,” and “patient,” used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Cancer,” “neoplasm,” “tumor,” “malignancy” and “carcinoma,” used interchangeably herein, refer to cells or tissues that exhibit an aberrant growth phenotype characterized by a significant loss of control of cell proliferation. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. The methods and compositions of this invention particularly apply to precancerous, malignant, pre-metastatic, metastatic, and non-metastatic cells.

The terms “treatment,” “treating,” “treat,” and the like refer to obtaining a desired pharmacological and/or physiologic effect in a tumor patient. The effect can be prophylactic in terms of completely or partially preventing tumor or symptom thereof and/or can be therapeutic in terms of a partial or complete stabilization or cure for tumor and/or adverse effect attributable to the tumor. Treatment covers any treatment of a tumor in a mammal, particularly a human. A desired effect, in particular, is tumor response, which can be measured as reduction of tumor mass or inhibition of tumor mass increase. In addition to tumor response, an increase of overall survival, progress-free survival, or time to tumor recurrence or a reduction of adverse effect also can be used clinically as a desired treatment effect.

As used herein, the term “administering” includes directly administering to another, self-administering, and prescribing or directing the administration of an agent as disclosed herein.

As used herein, the phrases “effective amount” and “therapeutically effective amount” mean that active agent dosage or plasma concentration in a subject, respectively, that provides the specific pharmacological effect for which the active agent is administered in a subject in need of such treatment. It is emphasized that an effective amount of an active agent will not always be effective in treating the conditions/diseases described herein, even though such dosage is deemed to be an effective amount by those of skill in the art.

As used herein, the term “active agent” is any small molecular drug, protein, functional nucleic acid, or polynucleic acid encoding a functional nucleic acid that is useful for treating a subject. The active agent can be any of the antineoplastic drugs, functional acids, interferon-type I agonists or type II agonists described herein.

The phrase “pharmaceutically acceptable” is employed herein to refer to those compounds, materials, compositions, and/or dosage forms which are, within the scope of sound medical judgment, suitable for use in vivo without excessive toxicity, irritation, allergic response, or other problem or complication, commensurate with a reasonable benefit/risk ratio. See further description below of various carriers, such as phosphate buffered saline (PBS) and sterile pyrogen-free water, that can qualify as pharmaceutically acceptable and hence suitable for use in accordance with the present invention.

Embodiments described herein are further illustrated by, though in no way limited to, the following prophetic examples.

### EXAMPLES

### Example 1. Method of Making a Composition Comprising a Pharmaceutically Acceptable Carrier and a Plurality of Delivery Elements Comprising a Bacterial α-Galactosylceramide Compound

The present Example illustrates an exemplary method of producing a composition of the present disclosure. In particular, illustrated is an exemplary method of producing a composition comprising (a) a pharmaceutically acceptable carrier; and (b) a plurality of intact bacterially derived minicells comprising an α-galactosylceramide (α-GalCer) compound that: (i) comprises (i) a N-acyl chain of 16 to 17 carbons in length and (ii) a hydroxyl group on the β-rather than the α-carbon; and (ii) does not present a 4-hydroxy group on its sphingoid base, wherein the compound can form a CD1d-lipid-TCR trimeric complex in vivo such that iNKT cells are activated. The α-GalCer compound is an α-GalCer_{B} compound.

Minicells are prepared as described herein. An α-galactosylceramide (α-GalCer) compound that: (a) comprises (i) a N-acyl chain of 16 to 17 carbons in length and (ii) a hydroxyl group on the β-rather than the α-carbon; and (b) does not present a 4-hydroxy group on its sphingoid base, is obtained. The α-GalCer compound is loaded into the minicells as described herein, thereby producing α-GalCer compound-packaged minicells. A composition comprising the α-GalCer compound-packaged minicells and a pharmaceutically acceptable carrier is produced. The α-GalCer compound is an α-GalCer_{B} compound.

Example 2. Method of making a dosage form comprising a single-use vial that comprises about 1×10¹⁰ to about 1×10¹¹ minicells packaged with an α-GalCer compound The present Example illustrates an exemplary method of producing a dosage form comprising a single-use vial comprising bacterial minicells packaged with an α-GalCer compound, such as an α-GalCer_{B} compound.

Minicells packaged with an α-galactosylceramide (α-GalCer) compound that: (a) comprises (i) a N-acyl chain of 16 to 17 carbons in length and (ii) a hydroxyl group on the β-rather than the α-carbon; and (b) does not present a 4-hydroxy group on its sphingoid base (α-GalCer compound-packaged minicells), are prepared as described in Example 1. About 1×10¹⁰ to about 1×10¹¹ α-GalCer compound-packaged minicells are added to a single-use vial, thereby producing a dosage form comprising a single-use vial that comprises about 1×10¹⁰ to about 1×10¹¹ minicells packaged with an α-GalCer compound. The α-GalCer compound compound is an α-GalCer_{B} compound.

### Example 3. Method of Treating an Infection

The present Example illustrates an exemplary method of treating an infection in a subject in accordance with the methods and compositions described in the present disclosure.

Minicells packaged with an α-galactosylceramide (α-GalCer) compound that: (a) comprises (i) a N-acyl chain of 16 to 17 carbons in length and (ii) a hydroxyl group on the β-rather than the α-carbon; and (b) does not present a 4-hydroxy group on its sphingoid base (α-GalCer compound-packaged minicells), are prepared as described in Example 1. The α-GalCer compound is an α-GalCer_{B} compound.

A subject having or suspected of having a viral or bacterial infection is treated for the viral or bacterial infection via a method involving administering to the subject a therapeutically effective amount of the α-GalCer compound-packaged minicells. The subject is a mammalian subject, such as a human subject.

A subject is vaccinated against the viral or bacterial infection via a method involving administering to the subject a therapeutically effective amount of the α-GalCer compound-packaged minicells. The subject is a mammalian subject, such as a human subject.

### Example 4. Method of Treating Cancer

The present Example illustrates an exemplary method of treating cancer in a subject in accordance with the methods and compositions described in the present disclosure.

Minicells packaged with an α-galactosylceramide (α-GalCer) compound that: (a) comprises (i) a N-acyl chain of 16 to 17 carbons in length and (ii) a hydroxyl group on the β-rather than the α-carbon; and (b) does not present a 4-hydroxy group on its sphingoid base (α-GalCer compound-packaged minicells), are prepared as described in Example 1. The α-GalCer compound is an α-GalCer_{B} compound.

A subject having or suspected of having cancer is treated via a method involving administering to the subject a therapeutically effective amount of the α-GalCer compound-packaged minicells. The subject is a mammalian subject, such as a human subject.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety. Further embodiments are set forth in the following claims.

### CITATIONS

Banchet-Cadeddu et al., Org. Biomolec. Chem. 3080(2011 )
Blauvelt et al., Bioorg. Med. Chem. Lett. 18: 6374(2008 )
Brahmbhatt & MacDiarmid, Microb Biotechnol., 15(1): 91-94 (2022)
Brown et al., Cell Host & Microbe 25: 668 (2019)
Chang et al., Proc. Nat'l Acad. Sci. USA 104: 10299 (2007)
Chiba et al., Arthritis Rheum. 50: 305(2004 )
Fujio et al., J. Am. Chem. Soc. 128: 9022(2006 )
Fung et al., Immunol. Rev. 260: 35(2014 )
Garg et al., Biochim. Biophys. Acta 1805: 53(2010 )
Gehrmann et al., Cancer Res. 73: 3865(2013 )
Giaccone et al., Clin. Cancer Res. 8: 3702(2002 )
Hayakawa et al., Eur. J. Immunol. 31: 1720(2001 )
Henson, Annu. Rev. Cell Devel. Biol. 33: 127(2017 )
Hung et al., Biochem. Pharmacol. 73: 1957(2007 )
Kaieda et al., Arthritis Rheum. 56: 1836(2007 )
Kim et al., Int'l J. Mol. Sci. 22: 14(2020 )
Kronenberg, M., Annu. Rev. Immunol. 23: 877-900 (2005)
Krysko et al., Trends Immunol. 32: 157(2011 )
Lang, S., “Production of Microbial Glycolipids,” in Bucke, C. (ed.) CARBOHYDRATE BIOTECHNOLOGY PROTOCOL. METHODS IN BIOTECHNOLOGY, vol. 10 (Humana Press, 1999)
Lee et al., J. Med. Chem. 50: 585(2007 )
Liang et al., J. Am. Chem. Soc. 130: 12348(2008)
Nakagawa et al., Cancer Res. 58: 1202(1998)
Padte et al., PLOS One 8: e78407 (2013)
Rock and Kono, Annu. Rev. Pathol. 3:99 (2008)
Savage et al., Chem. Soc. Rev. 35: 771(2006)
Schneiders et al. (2011a) In: NATURAL KILLER T CELLS. CANCER DRUG DISCOVERY AND DEVELOPMENT, Ch. 10 (Springer, NY 2012)
Schneiders et al., Clin. Immunol. 140: 130(2011b)
Thursby and Juge, Biochem. J. 474: 1823(2017)
Tsuji, Cell. Mol. Life Sci. 63: 1889(2006)
van Niel et al., Nat. Rev. Mol. Cell Bio. 19: 213-228 (2018)
Vartabedian, V. F. et al., Immunol. Rev. 272: 109-119 (2016)
von Gerichten et al., J. Lipid Res. 58: 1247(2017)
von Gerichten et al., J. Lipid Res. 60: 1892(2019)
Waldowska et al., Cent. Eur. J. Immunol. 42: 181(2017)
Wieland Brown et al., PLOS Biology 11: e1001610 (2013)
Zhu et al., PLOS Pathog. 11: e1004613 (2015)

1. A composition comprising:
(a) a pharmaceutically acceptable carrier; and
(b) a plurality of delivery elements selected from intact bacterially derived minicells, intact killed bacterial cells, intact mammalian cells, and extracellular vesicles, the delivery elements comprising an α-galactosylceramide compound that:
(i) comprises (i) a N-acyl chain of 16 to 17 carbons in length and (ii) a hydroxyl group on the β-rather than the α-carbon; and
(ii) does not present a 4-hydroxy group on its sphingoid base, wherein the compound can form a CD1d-lipid-TCR trimeric complex in vivo such that iNKT cells are activated.

2. The composition of claim 1, wherein the compound further comprises (iii) an iso-branched lipid terminus.

3. The composition of claim 1, wherein at least some of the delivery elements are mammalian cells selected from the group consisting of natural killer cells, dendritic cells, and macrophages.

4. The composition of claim 1, wherein at least some of the delivery elements are intact bacterially derived minicells.

5. The composition of claim wherein at least some of the delivery elements are extracellular vesicles.

6. The composition of claim 1, wherein the compound was purified to pharmaceutical grade after in vitro expression thereof by commensal bacteria.

7. The composition of claim 6, wherein the compound was purified to a level in a range from 97% to 99%.

8. The composition of claim 6, wherein the bacterium is selected from the group consisting of Bacteroides fragilis, Bacteroides vulgatus, and Prevotella copri.

9. The composition of claim 8, wherein the bacterium is Bacteroides fragilis.

10. The composition of claim 9, wherein the compound is α-GalCer_{Bf}.

11. A dosage form comprising a single-use vial that comprises about 1×10¹⁰ to about 1×10¹¹ minicells packaged with an α-GalCer compound as recited in claim 1, such that the vial comprises a therapeutically effective dose of the compound.

12. A method of treating or vaccinating against a viral or bacterial infection, comprising administering to a subject in need of such treating or vaccinating a therapeutically effective amount of a composition according to claim 1.

13. A method of treating cancer, comprising administering to a subject who suffers from a cancer pathology a therapeutically effective amount of a composition according to claim 1.

14. The composition of claim 8, wherein the bacterium is Bacteroides vulgatus.

15. The composition of claim 8, wherein the bacterium is Prevotella copri.
